(19) United States
(12) Patent Application Publication
(10) Pub. No.: US 2025/0375594 A1
(43) Pub. Date: Dec. 11, 2025

(54) Introducers and Assemblies Thereof with Tissue-Easing Transitions

(71) Applicant: Bard Access Systems, Inc., Salt Lake City, UT (US)

(72) Inventor: Eric W. Lindekugel, Salt Lake City, UT (US)

(21) Appl. No.: 18/738,485

(22) Filed: Jun. 10, 2024

(51) Int. Cl. A61M 25/06; A61M 25/01

(52) U.S. Cl. CPC A61M 25/0668; A61M 25/0606; A61M 2025/0183; A61M 2025/0687

(57) ABSTRACT

An introducer can include an introducer needle and an introducer sheath having a transition therebetween that eases tissue over a distal end of the introducer sheath without catching tissue thereon tissue despite an excised portion of the introducer needle. The introducer needle can include a needle shaft having a longitudinal needle slot extending from a proximal portion of the needle shaft through a distal needle tip, which results in an excised portion of a heel of a bevel of the needle tip. The introducer sheath can include a sheath body and a tapered sheath tip. The sheath body can be over the needle shaft sealing the needle slot thereunder but for a sheath-body opening in a proximal portion of the sheath body for an access guidewire. The introducer can include the transition from the bevel of the needle tip to at least the taper of the sheath tip.

### BACKGROUND

A central venous catheter (“CVCs”) is commonly introduced into a patient and advanced through his or her vasculature by way of the Seldinger technique. The Seldinger technique utilizes a number of steps and medical devices (e.g., a needle, a guidewire, a scalpel, a dilator, an introducer sheath, a CVC, etc.). While the Seldinger technique is effective, the number of steps are time consuming, handling the number of medical devices is awkward, and both of the foregoing can lead to patient trauma. In addition, there is a relatively high potential for touch contamination and subsequent infection due to the number of medical devices that need to be interchanged during the number of steps of the Seldinger technique. As such, there is a need to reduce the number of steps and medical devices involved in introducing a catheter such as a CVC into a patient and advancing the catheter through a vasculature thereof.

Efforts directed to the foregoing have led to the rapidly insertable central catheter (“RICC”), which utilizes an introducer having a slotted needle for rapidly deploying an access guidewire after establishing venous access with the introducer so as to maintain venous access upon withdrawing a remainder of the introducer. However, a slot of the slotted needle extends through a needle tip of the slotted needle as shown in FIGS. 10 and 11. This excises a portion of the needle tip, in particular, a heel portion of a bevel of the needle tip as further shown in FIGS. 10 and 11. Such an excised portion of the bevel can cause tissue (e.g., epidermal tissue, dermal tissue, hypodermal tissue, etc.) to bridge over the slot and catch on a distal end of an introducer sheath disposed over the slotted needle, which tissue requires additional insertion force to overcome during a venipuncture. That, and the tissue that catches on the distal end of the introducer sheath can be damaged by the additional insertion force. As such, there is a need to ease such tissue over the distal end of the introducer sheath from the needle tip of the slotted needle and reduce the foregoing insertion force.

Disclosed herein are introducers and assemblies thereof with tissue-easing transitions that address the foregoing.

### SUMMARY

Disclosed herein is an introducer including, in some embodiments, an introducer needle and an introducer sheath. The introducer needle includes a distal needle tip and a needle shaft. The needle tip includes a bevel. The needle shaft includes a longitudinal needle slot extending from a proximal portion of the needle shaft through the needle tip, thereby resulting in an excised portion of a heel of the bevel. The introducer sheath includes a sheath tip and a sheath body. The sheath tip includes a taper. The sheath body is over the needle shaft sealing the needle slot thereunder but for a sheath-body opening in a proximal portion of the sheath body. The introducer includes a transition from the bevel of the needle tip to at least the taper of the sheath tip. The transition is configured to ease tissue thereover without catching the tissue on a distal end of the sheath tip due to the excised portion of the heel of the bevel.

In some embodiments, the transition includes a longitudinal recess in the introducer sheath.

In some embodiments, the longitudinal recess in the introducer sheath extends through the taper of the sheath tip into a distal portion of the sheath body.

In some embodiments, a luminal side of the longitudinal recess includes a longitudinal protrusion of the introducer sheath that protrudes into the needle slot of the needle shaft.

In some embodiments, the longitudinal protrusion centers the introducer sheath over the introducer needle.

In some embodiments, the transition includes a transverse notch proximal of the excised portion of the heel of the bevel. At least a portion of the sheath tip is disposed in the notch.

In some embodiments, the distal end of the sheath tip is flush with a remaining portion of the heel of the bevel.

In some embodiments, the distal end of the sheath tip is subflush with a remaining portion of the heel of the bevel.

Also disclosed herein is an introducer assembly including, in some embodiments, an introducer and an access guidewire. The introducer includes an introducer needle and an introducer sheath. The introducer needle includes a distal needle tip and a needle shaft. The needle tip includes a bevel. The needle shaft includes a longitudinal needle slot extending from a proximal portion of the needle shaft through the needle tip, thereby resulting in an excised portion of a heel of the bevel. The introducer sheath includes a sheath tip and a sheath body. The sheath tip includes a taper. The sheath body is over the needle shaft sealing the needle slot thereunder but for a sheath-body opening in a proximal portion of the sheath body. The introducer includes a transition from the bevel of the needle tip to at least the taper of the sheath tip. The transition is configured to ease tissue thereover without catching the tissue on a distal end of the sheath tip due to the excised portion of the heel of the bevel. The access guidewire includes a distal end portion disposed in the introducer.

In some embodiments, the transition includes a longitudinal recess in the introducer sheath.

In some embodiments, the longitudinal recess in the introducer sheath extends through the taper of the sheath tip into a distal portion of the sheath body.

In some embodiments, a luminal side of the longitudinal recess includes a longitudinal protrusion of the introducer sheath that protrudes into the needle slot of the needle shaft.

In some embodiments, the longitudinal protrusion centers the introducer sheath over the introducer needle.

In some embodiments, the transition includes a transverse notch proximal of the excised portion of the heel of the bevel. At least a portion of the sheath tip is disposed in the notch.

In some embodiments, the distal end of the sheath tip is flush with a remaining portion of the heel of the bevel.

In some embodiments, the distal end of the sheath tip is subflush with a remaining portion of the heel of the bevel.

In some embodiments, the introducer assembly further includes a needle hub around a proximal end portion of the needle shaft.

In some embodiments, the introducer assembly further includes a syringe having a tapered male syringe tip extending from a distal portion of the syringe. The syringe tip is configured to insert into a tapered female needle-hub connector in a proximal portion of the needle hub.

In some embodiments, the introducer sheath further includes a splittable sheath hub around a proximal end portion of the sheath body.

Also disclosed herein is a RICC insertion assembly including, in some embodiments, a RICC, an introducer, and an access guidewire. The introducer includes an introducer needle and an introducer sheath. The introducer needle includes a distal needle tip and a needle shaft. The needle tip includes a bevel. The needle shaft includes a longitudinal needle slot extending from a proximal portion of the needle shaft through the needle tip, thereby resulting in an excised portion of a heel of the bevel. The introducer sheath includes a sheath tip and a sheath body. The sheath tip includes a taper. The sheath body is over the needle shaft sealing the needle slot thereunder but for a sheath-body opening in a proximal portion of the sheath body. The introducer includes a transition from the bevel of the needle tip to at least the taper of the sheath tip. The transition is configured to ease tissue thereover without catching the tissue on a distal end of the sheath tip due to the excised portion of the heel of the bevel. The access guidewire includes a proximal portion disposed in the RICC and a distal end portion disposed in the introducer.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a RICC insertion assembly in accordance with some embodiments.

FIG. 2 illustrates a first view of a distal portion of the RICC insertion assembly in accordance with some embodiments.

FIG. 3 illustrates a second view of the distal portion of the RICC insertion assembly in accordance with some embodiments.

FIG. 4 illustrates the introducer without a sheath hub of the introducer sheath for expository expediency.

FIG. 5 illustrates the introducer needle including a needle shaft and a needle hub in accordance with some embodiments.

FIG. 6 illustrates the introducer sheath including a sheath body and the sheath hub in accordance with some embodiments.

FIG. 7 illustrates a transition from a bevel of a needle tip of the introducer needle to at least a taper of a sheath tip of the introducer sheath in accordance with some embodiments.

FIG. 8 illustrates another transition from the bevel of the needle tip to at least the taper of the sheath tip in accordance with some embodiments.

FIG. 9 illustrates the transition from the bevel of the needle tip to the taper of the sheath tip of FIG. 8 but with the introducer sheath in place over the introducer needle in accordance with some embodiments.

FIG. 10 illustrates an isometric view of a distal end portion of an existing introducer for a RICC.

FIG. 11 illustrates an end view of the distal end portion of the introducer of FIG. 10.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

Unless defined otherwise, all technical and scientific terms used herein have the same meaning as commonly understood by those of ordinary skill in the art.

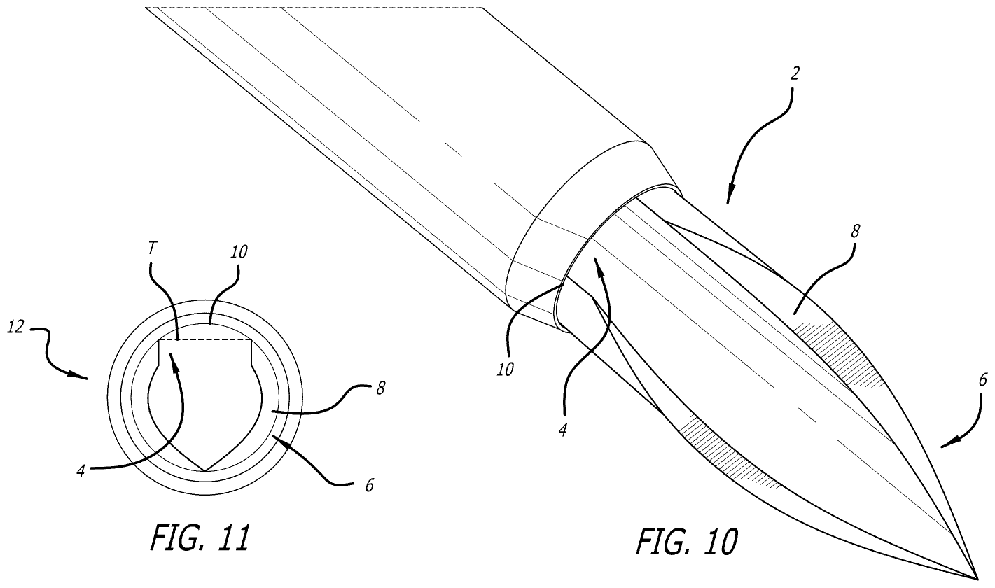

As set forth above, efforts directed to reduce the number of steps and medical devices involved in introducing a catheter such as a CVC into a patient and advancing the catheter through the vasculature thereof have led to the RICC, which utilizes an introducer having a slotted needle 2 for rapidly deploying an access guidewire after establishing venous access with the introducer so as to maintain venous access upon withdrawing a remainder of the introducer. However, a slot 4 of the slotted needle 2 extends through a needle tip 6 of the slotted needle 2 as shown in FIGS. 10 and 11. This excises a portion of the needle tip 6, in particular, a heel portion of a bevel 8 of the needle tip 6 as further shown in FIGS. 10 and 11. Such an excised portion of the bevel 8 can cause tissue T (e.g., epidermal tissue, dermal tissue, hypodermal tissue, etc.) to bridge over the slot 4 and catch on a distal end 10 of an introducer sheath 12 disposed over the slotted needle 2, which tissue T requires additional insertion force to overcome during a venipuncture. That, and the tissue T that catches on the distal end 10 of the introducer sheath 12 can be damaged by the additional insertion force. As such, there is a need to case such tissue T over the distal end of the introducer sheath 12 from the needle tip 6 of the slotted needle 2 and reduce the foregoing insertion force.

Disclosed herein are introducers and assemblies thereof with tissue-easing transitions that address the foregoing.

### RICC Insertion Assemblies

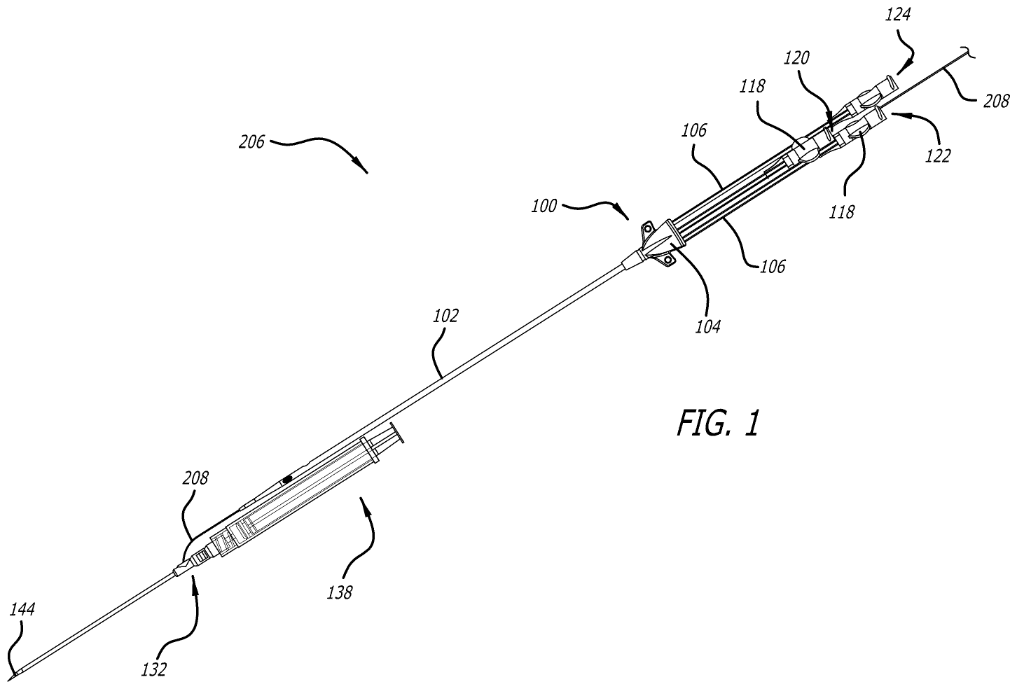

FIGS. 1-3 illustrate different views of a RICC insertion assembly 206 in accordance with some embodiments.

As shown, the RICC insertion assembly 206 includes a RICC 100, an introducer 132, optionally, including a syringe 138, and an access guidewire 208 disposed in both the RICC 100 and the introducer 132 in at least a ready-to-operate state of the RICC insertion assembly 206. Notably, in the ready-to-operate state of the RICC insertion assembly 206, a proximal portion of the access guidewire 208 is disposed in the RICC 100, and a distal end portion of the access guidewire 208 is disposed in the introducer 132, each of the foregoing components of the RICC insertion assembly 206 thereby connected to at least one other component of the RICC insertion assembly 206.

Description for each of the RICC 100, the introducer 132, and the access guidewire is set forth below. Notably, various configurations of the introducer needle 134 and the introducer sheath 136 for easing tissue over the distal end of the introducer sheath 136 from the needle tip 144 of the introducer needle 134 are described with respect to the introducer 132.

### RICCs

FIGS. 1-3 also illustrate the RICC 100 or portions thereof in accordance with some embodiments.

As shown, the RICC 100 includes the catheter tube 102, a catheter hub 104, and one or more extension legs 106.

The catheter tube 102 includes a first section 108 in the distal portion of the catheter tube 102, a second section 110 in the distal portion of the catheter tube 102 proximal of the first section 108, and a tapered junction 112 between the first and second sections 108 and 110 of the catheter tube 102.

The first section 108 of the catheter tube 102 includes a distal tip 114 having a relatively short taper from an outer diameter of a distal end of the first section 108 to an outer diameter of a remainder of the first section 108. The taper of the distal tip 114 is configured for immediate dilation of tissue about a needle tract established with the introducer 132 up to the outer diameter of the remainder of the first section 108 of the catheter tube 102. The second section 110 of the catheter tube 102 includes a consistent outer diameter over its length from a distal end of the second section 110 to a proximal end of the second section 110. The consistent diameter of the second section 110 of the catheter tube 102 is configured for smooth insertion into the needle tract and targeted vasculature subsequent to any dilation by the first section 108 of the catheter tube 102 and the junction 112. The junction 112 includes a taper over its length from a distal end of the junction 112 to the proximal end of the junction 112. The taper of the junction 112 is configured for immediate dilation of the tissue about the needle tract from the outer diameter of a majority of the first section 108 of the catheter tube 102 to the outer diameter of the second section 110 of the catheter tube 102.

The first section 108 of the catheter tube 102 is formed of a first polymeric material (e.g., a polytetrafluoroethylene, a polypropylene, or a polyurethane) having a first durometer. The second section 110 of the catheter tube 102 is formed of a second polymeric material (e.g., a polyvinyl chloride, a polyethylene, another polyurethane, or a silicone) having a second durometer less than the first durometer. For example, the first section 108 of the catheter tube 102 can be formed of a first polyurethane having the first durometer while the second section 110 of the catheter tube 102 can be formed of a second, different polyurethane (e.g., a same or different diisocyanate or triisocyanate reacted with a different diol or triol, a different diisocyanate or triisocyanate reacted with a same or different diol or triol, etc.) having the second durometer less than the first durometer. Indeed, polyurethanes are advantageous for the catheter tube 102 in that polyurethanes can be relatively rigid at room-temperature but become more flexible in vivo at body temperature, which reduces irritation to vessel walls and phlebitis. Polyurethanes are also advantageous in that they can be less thrombogenic than some other polymers. The junction 112 is formed of the second polymeric material or a third polymeric material (e.g., yet another polyurethane) having a third durometer less than the first durometer and greater than, approximately equal to, or less than the second durometer.

It should be understood the first durometer of the first polymeric material, the second durometer of the second polymeric material, and the third durometer of the third polymeric material can be on different scales (e.g., Type A or Type D). With this understanding, the second durometer of the second polymeric material or the third durometer of the third polymeric material might not be numerically less than the first durometer of the first polymeric material when the second durometer or the third durometer is less than the first durometer. Indeed, the hardness of the second polymeric material or the third polymeric material can still be less than the hardness of the first polymeric material as the different scales—each of which ranges from 0 to 100—are designed for characterizing different materials in groups of the materials having a like hardness.

In accordance with the first section 108 of the catheter tube 102, the second section 110 of the catheter tube 102, and the junction 112 between the first and second sections 108 and 110 of the catheter tube 102 set forth above, the catheter tube 102 possesses a column strength sufficient to prevent buckling of the catheter tube 102 when inserted into a needle tract established by a percutaneous puncture with the introducer 132. The column strength of the catheter tube 102 is also sufficient to prevent buckling of the catheter tube 102 when advanced through a vasculature of a patient without dilation of tissue about the needle tract or any blood vessels of the vasculature beforehand with a separate dilator.

The catheter tube 102 includes one or more catheter-tube lumens extending through the catheter tube 102; however, only one catheter-tube lumen typically extends from a proximal end of the catheter tube 102 to a distal end of the catheter tube 102 in a multiluminal RICC (e.g., a diluminal RICC, a triluminal RICC, a tetraluminal RICC, a pentaluminal RICC, a hexaluminal RICC, etc.).

The catheter hub 104 is coupled to a proximal portion of the catheter tube 102. The catheter hub 104 includes one or more catheter-hub lumens corresponding in number to the one-or-more catheter-tube lumens. The one-or-more catheter-hub lumens extends through an entirety of the catheter hub 104 from a proximal end of the catheter hub 104 to a distal end of the catheter hub 104.

Each extension leg of the one-or-more extension legs 106 is coupled to the catheter hub 104 by a distal portion thereof. The one-or-more extension legs 106 respectively include one or more extension-leg lumens, which, in turn, correspond in number to the one-or-more catheter-hub lumens. Each extension-leg lumen of the one-or-more extension-leg lumens extends through an entirety of the extension leg from a proximal end of the extension leg to a distal end of the extension leg.

Each extension leg of the one-or-more extension legs 106 includes a Luer connector 118 coupled to the extension leg, through which Luer connector 118 the extension leg and the extension-leg lumen thereof can be connected to another medical device and a lumen thereof.

As shown, the RICC 100 is a triluminal RICC including a set of three lumens; however, the RICC 100 is not limited to the set of the three lumens. The set of three lumens includes a primary lumen 120, a secondary lumen 122, and a tertiary lumen 124 formed of fluidly connected portions of three catheter-tube lumens, three catheter-hub lumens, and three extension-leg lumens. The primary lumen 120 has a primary-lumen aperture in the distal end of the first section 108 of the catheter tube 102, which corresponds to the distal end of the catheter tube 102 and a distal end of the RICC 100. While not expressly shown, the primary-lumen aperture is that from which the access guidewire 208 extends in FIGS. 1-3. The secondary lumen 122 has a secondary-lumen aperture 128 in a side of the distal portion of the catheter tube 102. The tertiary lumen 124 has a tertiary-lumen aperture 130 in the side of the distal portion of the catheter tube 102 proximal of the secondary-lumen aperture 128.

### Introducers

FIGS. 1-3 also illustrate the introducer 132 in accordance with some embodiments.

As shown, the introducer 132 includes an introducer needle 134, a splittable introducer sheath 136, and optionally, the syringe 138 fluidly connected to a combination of the foregoing. Indeed, the introducer sheath 136 is disposed over the introducer needle 134 in at least the ready-to-operate state of the introducer 132 for establishing a needle tract, and the syringe 138 is fluidly connected to the needle hub 142 for aspirating blood with the syringe 138 after establishing the needle tract.

FIG. 5 illustrates the introducer needle 134 of the introducer 132 in accordance with some embodiments.

The introducer needle 134 includes a needle shaft 140 and a needle hub 142 coupled to a proximal portion of the needle shaft 140, particularly a proximal end portion of the needle shaft 140.

The needle shaft 140 includes a distal needle tip 144 in a distal portion of the needle shaft 140 and a longitudinal needle slot 146.

The needle tip 144 includes a bevel 150 extending substantially up to an entirety of a length of the needle tip 144. Such a needle tip is configured for establishing a needle tract from an area of skin into a blood-vessel lumen of a patient.

The needle slot 146 distally extends from a proximal portion of the needle shaft 140 through the needle tip 144, thereby forming a needle channel along a majority of a length of the introducer needle 134. In addition, extension of the needle slot 146 from the proximal portion of the needle shaft 140 through the needle tip 144 results in an excised portion of a heel of the bevel 150 as set forth above. The needle slot 146 has a width sized in accordance with the outer diameter of the access guidewire 208. Notably, the introducer 132 includes an introducer lumen 152, which is formed by a combination of the introducer needle 134 and the introducer sheath 136, particularly, from the introducer sheath 136 over the needle channel of the introducer needle 134 as in the ready-to-operate state of the introducer 132. Indeed, the introducer sheath 136 seals the needle slot 146 thereunder forming the introducer lumen 152 from the needle channel of the introducer needle 134.

The needle hub 142 can include a non-tapered but optionally chamfered male needle-hub connector 156 in a distal portion of the needle hub 142 as well as a tapered female needle-hub connector 158 in a proximal portion of the needle hub 142. The male needle-hub connector 156 is configured to form a fluid-tight connection with the female sheath-hub connector 182 set forth below in at least the ready-to-operate state of the introducer 132. Indeed, the male needle-hub connector 156 can include an ‘O’-ring 160 disposed in a circumferential groove around the male needle-hub connector 156 configured to form the fluid-tight connection. The female needle-hub connector 158 is configured to form a fluid-tight connection with the syringe tip 204 of the syringe 138 set forth below.

FIG. 6 illustrates the introducer sheath 136 of the introducer 132 in accordance with some embodiments.

The introducer sheath 136 includes a splittable sheath body 162 and a splittable sheath hub 164 around a proximal portion of the sheath body 162 and coupled thereto. The introducer sheath 136 is configured such that an entirety of the introducer sheath 136 is disposed over the introducer needle 134 in at least the ready-to-operate state of the introducer 132. Indeed, an entirety of the sheath body 162 is disposed over the needle shaft 140 and the sheath hub 164 is coupled to the proximal portion of the sheath body 162 such that the male needle-hub connector 156 of the needle hub 142 forms a fluid-tight connection with the female sheath-hub connector 182 of the sheath hub 164 in the ready-to-operate state of the introducer 132.

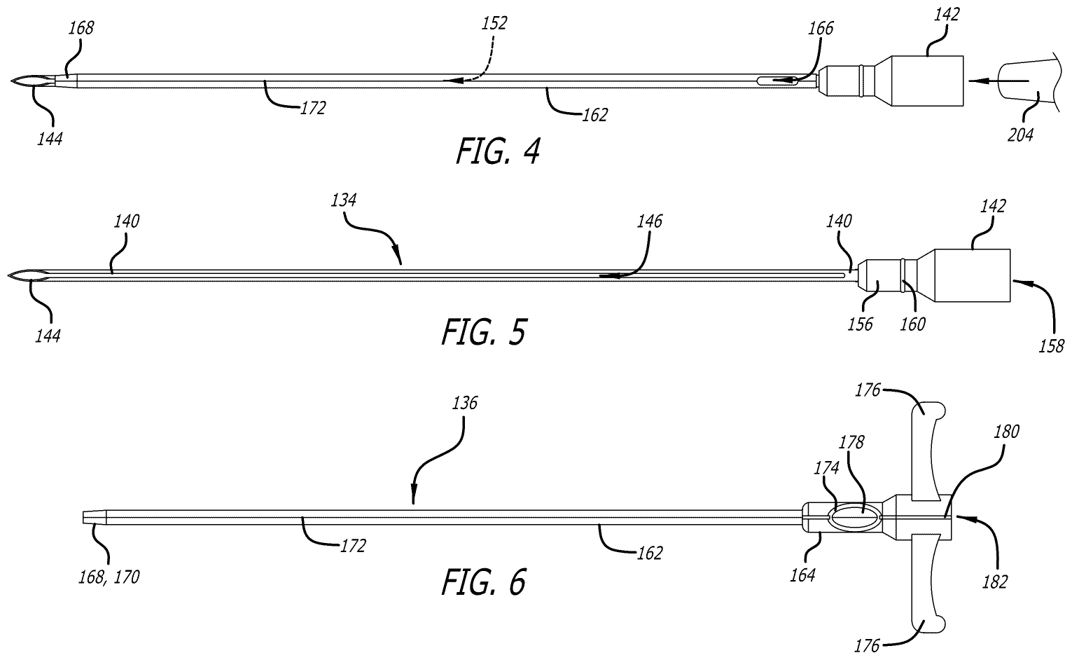

The sheath body 162 includes a sheath-body opening 166 in the proximal portion of the sheath body 162 and a sheath tip 168 in a distal portion of the sheath body 162. (See FIG. 4 for the sheath-body opening 166.) Notwithstanding the sheath-body opening 166, which is covered by the sheath hub 164 coupled to the proximal portion of the sheath body 162, the sheath body 162 is configured to seal the needle shaft 140 thereunder such that a vacuum can be drawn through the introducer 132 in at least the ready-to-operate state of the introducer 132.

The sheath-body opening 166 has a width approximately commensurate with a width of the needle slot 146 in the needle shaft 140, which, as set forth above, can be sized in accordance with the outer diameter of the access guidewire 208. The sheath-body opening 166 is thusly configured to allow the access guidewire 208 to pass therethrough and into the needle slot 146 of the needle shaft 140.

The sheath tip 168 includes a taper 170 from an outer diameter of the needle shaft 140 up to an outer diameter of a majority of the sheath body 162. Said differently, the taper 170 is from the outer diameter of the majority of the sheath body 162 down to the outer diameter of the needle shaft 140. The sheath tip 168 including such a taper is configured to provide a smooth transition from a distal end of the sheath tip 168 to the sheath body 162 in the ready-to-operate state of the introducer 132.

The sheath body 162 can also include one or more longitudinal body faults 172 (e.g., grooves, lines of weakened material, etc.) such as a pair of the body faults 172 extending along opposite sides of the sheath body 162 if the sheath body 162 is not formed of a material configured to split or propagate a split. For example, the sheath body 162 can be formed of a polymeric material such as polytetrafluoroethylene, which facilitates smooth, consistent splitting of the sheath body 162 without the body faults 172 along the sides of the sheath body 162. When present, the one-or-more body faults 172 include at least a primary body fault extending along at least a portion of a primary side of the introducer sheath 136 including the port 174. Indeed, the primary body fault can extend along an entirety of the sheath body 162 as shown in FIG. 4. When the pair of the body faults 172 is present, a secondary body fault extends along at least a portion of a secondary side of the introducer sheath 136 opposite the primary side. While not shown, the secondary body fault can also extend along the entirety of the sheath body 162.

The sheath hub 164 includes a splittable valved port 174 in the primary side of the introducer sheath 136 and a pair of tabs 176 extending from the sheath hub 164 proximal of the port 174.

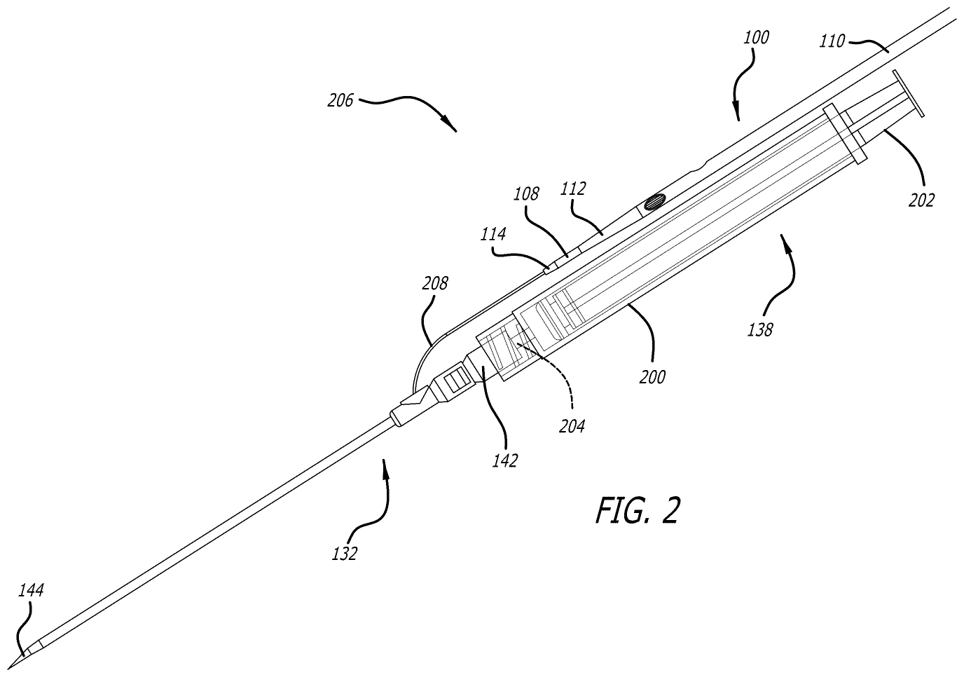

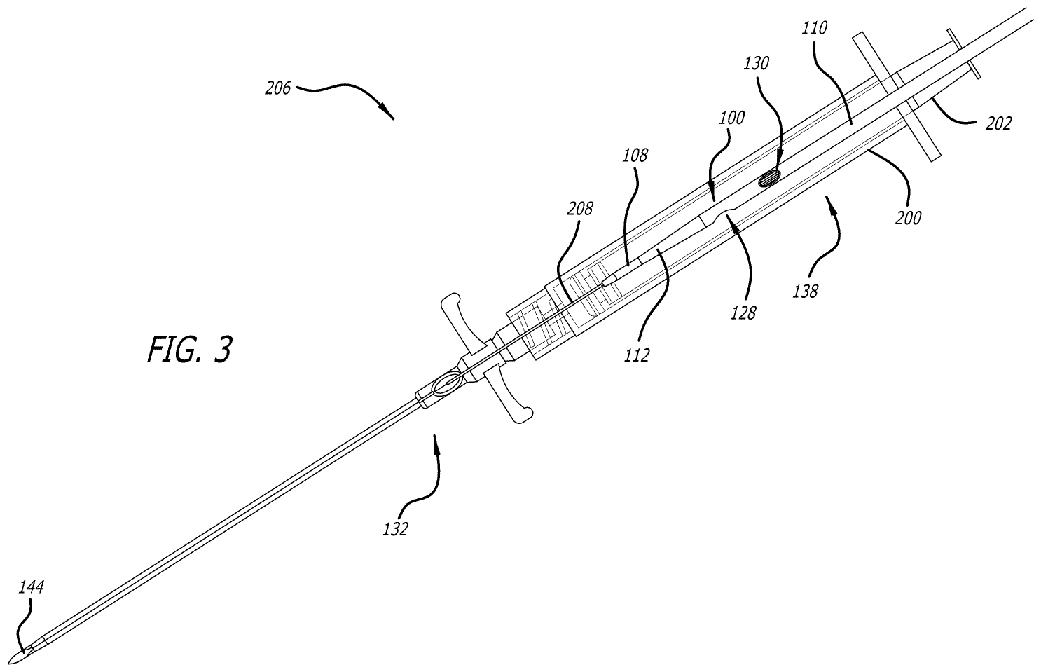

The port 174 lies over the sheath-body opening 166 in the proximal portion of the sheath body 162. While the port 174 has a length approximately commensurate with a length of the sheath-body opening 166, a width of the port 174 is wider than the width of the sheath-body opening 166 to accommodate a valve 178 such as a partially or fully split septum compressed in the port 174 over the sheath-body opening 166 of the sheath body 162. As best shown in FIGS. 2 and 3, the valve 178 is configured to allow the access guidewire 208 to pass through the valve 178, through the port 174, and into the sheath-body opening 166 of the sheath body 162, notably, while maintaining fluid-tight access to a blood-vessel lumen of a patient with the introducer 132 and, thereby, enabling blood-aspiration with the syringe 138. Such a valve is also configured to split with a remainder of the sheath hub 164 when splitting the introducer sheath 136 away from the access guidewire 208.

The tabs 176 radially extend from opposite sides of the sheath hub 164 orthogonal to the primary and secondary sides of the introducer sheath 136, which, in turn, positions the tabs 176 orthogonal to at least the primary body fault when present in the primary side of the introducer sheath 136. Such tabs are configured to split the sheath hub 164 when the tabs 176 are pulled apart from each other.

The sheath hub 164 can also include a pair of longitudinal hub faults 180 (e.g., grooves, lines of weakened material, etc.) extending along opposing sides of the sheath hub 164. When present, the hub faults 180 include a primary hub fault and a secondary hub fault. The primary hub fault extends along at least a portion of the primary side of the introducer sheath 136 (i.e., the side of the introducer sheath 136 including the port 174). Indeed, the primary hub fault can extend along an entirety of the sheath hub 164 as shown in FIG. 6. While not shown, the secondary hub fault extends along at least a portion of the secondary side of the introducer sheath 136 opposite the primary side. The sheath hub 164 is configured to split along both the primary and secondary hub faults 180 when the tabs 176 are pulled apart (or pinched together) for propagating splits resulting from splitting the sheath hub 164 along the sheath body 162 in the primary and secondary sides of the introducer sheath 136.

The sheath hub 164 can also include a non-tapered female sheath-hub connector 182 in a proximal portion of the sheath hub 164. The female sheath-hub connector 182 is configured to form a fluid-tight connection with the male needle-hub connector 156 in at least the ready-to-operate state of the introducer 132. Such non-tapered connectors are less likely to get stuck together than tapered connectors (e.g., Luer-tapered connectors), which is advantageous when withdrawing the introducer needle 134 from the introducer 132 and leaving the introducer sheath 136 in place in a blood-vessel lumen of a patient.

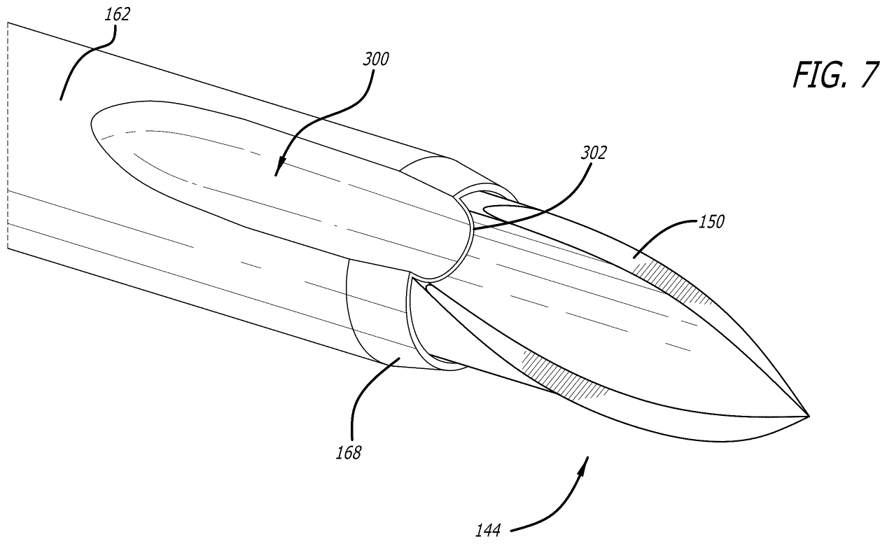

FIGS. 7-9 illustrate transitions from the bevel 150 of the needle tip 144 of the introducer needle 134 to at least the taper 170 of the sheath tip 168 of the introducer sheath 136 in accordance with some embodiments. Such transitions, alone or in combination with each other, case the tissue T over the distal end of the introducer sheath 136 from the needle tip 144 of the introducer needle 134, notably, without substantially catching the tissue T as with other introducers having the excised portion of the heel of the bevel 150. In addition, such transitions advantageously reduce the insertion force required for venipunctures.

As shown in FIG. 7, the transition can include a longitudinal recess 300 in the introducer sheath 136, particularly, in an abluminal side of the introducer sheath 136. The longitudinal recess 300 in the introducer sheath 136 can extend through the taper 170 of the sheath tip 168 into a distal portion of the sheath body 162. A luminal side of the longitudinal recess 300 can complement the abluminal side, the luminal side of the longitudinal recess 300 forming a longitudinal protrusion 302 of the introducer sheath 136 that protrudes into the needle slot 146 of the needle shaft 140. Advantageously, the longitudinal protrusion 302 can center the introducer sheath 136 over the introducer needle 134 such as during manufacturing, thereby aligning the body faults 172 of the introducer sheath 136, if present, with the needle slot 146 of the introducer needle 134 for cleanly releasing the access guidewire 208 from a top or clinician-facing side the introducer sheath 136 when the introducer sheath 136 is split away from the access guidewire 208.

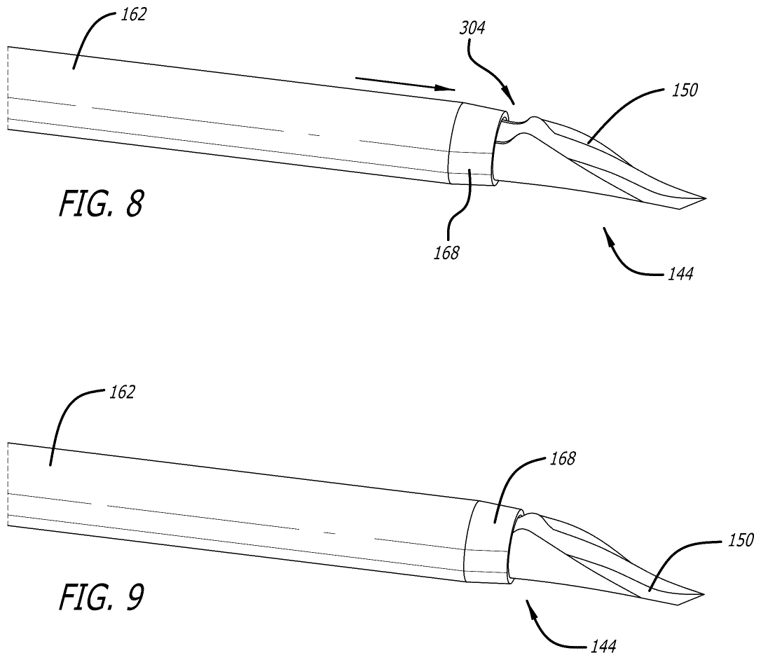

As shown in FIG. 8, the transition can include a transverse notch 304 proximal of the excised portion of the heel of the bevel 150. At least a portion of the sheath tip 168 can be disposed in the notch 304 as shown in FIG. 9 when the introducer sheath 136 is properly placed over the introducer needle 134. When the introducer sheath 136 is in place over the introducer needle 134, the distal end of the sheath tip 168 can be flush or subflush with a remaining portion of the heel of the bevel 150. Notably, the notch 304 can instead be circumferential, thereby resembling a distalmost barb of a barbed hose fitting. Akin to that shown in FIG. 9, at least a portion of the sheath tip 168 can be flushly or subflushly disposed in such a circumferential notch when the introducer sheath 136 is properly placed over the introducer needle 134. Whether the transition is the transverse notch 304 or the foregoing circumferential variant, it can be cut by way of a Computer Numerical Control (“CNC”) laser.

Lastly, the syringe 138 includes a barrel 200, a plunger 202 disposed in the barrel 200, and a tapered male syringe tip 204 extending from a distal portion of the syringe 138. The syringe tip 204 is configured to insert into the female needle-hub connector 158 set forth above. Such a syringe is useful for at least aspirating blood after establishing a needle tract with the introducer 132.

### Access Guidewire

The access guidewire 208 extends along an entirety of the primary lumen 120 of the RICC 100, through the valve 178 (e.g., the septum) of the port 174 of the sheath hub 164 of the introducer sheath 136, along the introducer lumen of the introducer 132, and to a location in the introducer 132 proximal of the needle tip 144 in at least a ready-to-operate state of the RICC insertion assembly 206. The location of the access guidewire 208 proximal of the needle tip 144 is advantageous for immediate advancement of a distal end of the access guidewire 208 through a remainder of the introducer 132 and into a blood-vessel lumen after establishing the needle tract.

### Methods

Methods include at least a method for inserting the RICC 100 into a blood-vessel lumen of a patient. Such a method includes one or more steps selected from an insertion assembly-acquiring step, a needle tract-establishing step, a blood-aspirating step, an access guidewire-advancing step, an introducer needle-withdrawing step, an introducer sheath-splitting step, a RICC-advancing step, and an access guidewire-withdrawing step.

The insertion assembly-acquiring step includes acquiring the RICC insertion assembly 206 including the RICC 100, the introducer 132, and the access guidewire 208 disposed in both the RICC 100 and the introducer 132. Optionally, the RICC insertion assembly 206 is acquired in a substantially ready-to-operate state of the RICC insertion assembly 206. If the RICC insertion assembly 206 is not acquired in the substantially ready-to-operate state thereof, the RICC insertion assembly 206 can be adjusted as needed to put the RICC insertion assembly 206 in the ready-to-operate state thereof before performing the needle tract-establishing step.

The needle tract-establishing step includes establishing a needle tract from an area of skin to the blood-vessel lumen with the introducer 132. As set forth above, transitions from the bevel 150 of the needle tip 144 to at least the taper 170 of the sheath tip 168 such as those shown in FIGS. 7-9 case the tissue T over the distal end of the introducer sheath 136 from the needle tip 144 of the introducer needle 134, notably, without substantially catching the tissue T as with other introducers having the excised portion of the heel of the bevel 150. In addition, such transitions advantageously reduce the insertion force required for the needle tract-establishing step.

Notably, the needle tract-establishing step can include drawing a slight vacuum with the syringe 138 while establishing the needle tract such that blood flashes back into at least the introducer needle 134 upon establishing the needle tract. In accordance with drawing such a vacuum, the needle tract-establishing step can include ensuring blood flashes back along the needle slot 146 of the introducer needle 134 if the sheath body 162 is formed of a clear polymeric material, into the needle hub 142 of the introducer needle 134 if the needle hub 142 is formed of a clear polymeric material, into the syringe tip 204, into the barrel 200 of the syringe 138, or a combination thereof, thereby confirming the needle tract extends into the blood-vessel lumen.

The blood-aspirating step includes aspirating blood with the syringe 138 coupled to the introducer needle 134 for confirmation the needle tract extends into the blood-vessel lumen at any time before withdrawing the introducer needle 134 from the introducer sheath 136 in the introducer needle-withdrawing step. Notably, the needle shaft 140 has an inner diameter sized in accordance with the outer diameter of the distal end portion of the access guidewire 208 to allow blood flashback and aspiration. This advantageously enables the blood-aspirating step to be performed even after the following access guidewire-advancing step whereby the access guidewire 208 secures or maintains the blood-vessel access for the RICC-advancing step.

The access guidewire-advancing step includes advancing the distal end of the access guidewire 208 from its initial location in the introducer 132 proximal of the needle tip 144 into the blood-vessel lumen to secure blood-vessel access for the RICC-advancing step. The remainder of the access guidewire 208 proximally extends along the introducer lumen of the introducer 132, through the valve 178 of the port 174 in the side of the sheath hub 164, along the entirety of the primary lumen 120 of the RICC 100, and out a proximal end of the RICC 100.

The introducer needle-withdrawing step includes withdrawing the introducer needle 134 from the introducer sheath 136 leaving both the introducer sheath 136 and the access guidewire 208 in place in the blood-vessel lumen. Again, the introducer needle 134 includes the needle slot 146 extending from the proximal portion of the needle shaft 140 through the needle tip 144, thereby allowing removal of the introducer needle 134 from the introducer sheath 136 while the access guidewire 208 remains in place in the blood-vessel lumen.

The introducer sheath-splitting step includes splitting the introducer sheath 136 away from the access guidewire 208 to remove split portions of the introducer sheath 136 from the blood-vessel lumen. Such splitting includes splitting the sheath hub 164 and the port 174 thereof by pulling apart the tabs 176 extending from the sheath hub 164. The introducer sheath-splitting step also includes propagating splits resulting from the splitting of the sheath hub 164 along the sheath body 162 of the introducer sheath 136. In this way, the introducer sheath 136 is split away from the access guidewire 208 to remove the split portions of the introducer sheath 136 from the blood-vessel lumen.

The RICC-advancing step includes advancing the distal portion of the catheter tube 102 of the RICC 100 over the access guidewire 208 and into the blood-vessel lumen, thereby inserting the RICC 100 into the blood-vessel lumen.

The access guidewire-withdrawing step includes withdrawing the access guidewire 208 leaving the catheter tube 102 in place in the blood-vessel lumen. The access guidewire-withdrawing step also includes removing the access guidewire 208 from the primary lumen 120 of the RICC 100 so a maneuver guidewire-advancing step can be performed.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly. departures may be made from the particular embodiments disclosed herein without departing from the scope of the concepts provided herein.

What is claimed is:

1. An introducer, comprising:
an introducer needle including:
a distal needle tip including a bevel; and
a needle shaft including a longitudinal needle slot extending from a proximal portion of the needle shaft through the needle tip, thereby resulting in an excised portion of a heel of the bevel; and
an introducer sheath including:
a sheath tip including a taper; and
a sheath body over the needle shaft sealing the needle slot thereunder but for a sheath-body opening in a proximal portion of the sheath body, the introducer including a transition from the bevel of the needle tip to at least the taper of the sheath tip, and the transition configured to ease tissue thereover without catching the tissue on a distal end of the sheath tip due to the excised portion of the heel of the bevel.

2. The introducer of claim 1, wherein the transition includes a longitudinal recess in the introducer sheath.

3. The introducer of claim 2, wherein the longitudinal recess in the introducer sheath extends through the taper of the sheath tip into a distal portion of the sheath body.

4. The introducer of claim 2, wherein a luminal side of the longitudinal recess includes a longitudinal protrusion of the introducer sheath that protrudes into the needle slot of the needle shaft.

5. The introducer of claim 4, wherein the longitudinal protrusion centers the introducer sheath over the introducer needle.

6. The introducer of claim 1, wherein the transition includes a transverse notch proximal of the excised portion of the heel of the bevel, at least a portion of the sheath tip disposed in the notch.

7. The introducer of claim 6, wherein the distal end of the sheath tip is flush with a remaining portion of the heel of the bevel.

8. The introducer of claim 6, wherein the distal end of the sheath tip is subflush with a remaining portion of the heel of the bevel.

9. An introducer assembly, comprising:
an introducer including:
an introducer needle including:
a distal needle tip including a bevel; and
a needle shaft including a longitudinal needle slot extending from a proximal portion of the needle shaft through the needle tip, thereby resulting in an excised portion of a heel of the bevel; and
an introducer sheath including:
a sheath tip including a taper; and
a sheath body over the needle shaft sealing the needle slot thereunder but for a sheath-body opening in a proximal portion of the sheath body, the introducer including a transition from the bevel of the needle tip to at least the taper of the sheath tip, and the transition configured to ease tissue thereover without catching the tissue on a distal end of the sheath tip due to the excised portion of the heel of the bevel; and
an access guidewire including a distal end portion disposed in the introducer.

10. The introducer assembly of claim 9, wherein the transition includes a longitudinal recess in the introducer sheath.

11. The introducer assembly of claim 10, wherein the longitudinal recess in the introducer sheath extends through the taper of the sheath tip into a distal portion of the sheath body.

12. The introducer assembly of claim 10, wherein a luminal side of the longitudinal recess includes a longitudinal protrusion of the introducer sheath that protrudes into the needle slot of the needle shaft.

13. The introducer assembly of claim 12, wherein the longitudinal protrusion centers the introducer sheath over the introducer needle.

14. The introducer assembly of claim 9, wherein the transition includes a transverse notch proximal of the excised portion of the heel of the bevel, at least a portion of the sheath tip disposed in the notch.

15. The introducer assembly of claim 14, wherein the distal end of the sheath tip is flush with a remaining portion of the heel of the bevel.

16. The introducer assembly of claim 14, wherein the distal end of the sheath tip is subflush with a remaining portion of the heel of the bevel.

17. The introducer assembly of claim 9, further comprising a needle hub around a proximal end portion of the needle shaft.

18. The introducer assembly of claim 17, further comprising a syringe including a tapered male syringe tip extending from a distal portion of the syringe, the syringe tip configured to insert into a tapered female needle-hub connector in a proximal portion of the needle hub.

19. The introducer assembly of claim 9, the introducer sheath further including a splittable sheath hub around a proximal end portion of the sheath body.

20. A rapidly insertable central catheter (“RICC”) insertion assembly, comprising:
a RICC;
an introducer including:
an introducer needle including:
a distal needle tip including a bevel; and
a needle shaft including a longitudinal needle slot extending from a proximal portion of the needle shaft through the needle tip, thereby resulting in an excised portion of a heel of the bevel; and
an introducer sheath including:
a sheath tip including a taper; and
a sheath body over the needle shaft sealing the needle slot thereunder but for a sheath-body opening in a proximal portion of the sheath body, the introducer including a transition from the bevel of the needle tip to at least the taper of the sheath tip, and the transition configured to case tissue thereover without catching the tissue on a distal end of the sheath tip due to the excised portion of the heel of the bevel; and
an access guidewire including proximal portion disposed in the RICC and a distal end portion disposed in the introducer.
